(19) United States
(12) Patent Application Publication
(10) Pub. No.: US 2010/0068307 A1
(43) Pub. Date: Mar. 18, 2010

(54) DERMATOLOGICAL EMULSION AND METHOD FOR THE PREPARATION THEREOF

(75) Inventor: Francoise Nielloud, Castelnau Le Lez (FR)

Correspondence Address: BIRCH STEWART KOLASCH & BIRCH, PO BOX 747, FALLS CHURCH, VA, 22040-0747, US

(73) Assignee: PIERRE FABRE DERMO-COSMETIQUE, Boulogen-Billancourt (FR)

(21) Appl. No.: 12/523,263

(22) PCT Filed: Jan. 25, 2008
(86) PCT No.: PCT/EP2008/050879

§ 371 Date: Jul. 15, 2009

(30) Foreign Application Priority Data
Jan. 26, 2007 (FR) 07/52901

(51) Int. Cl. A61K 33/04; A61K 31/57; A61K 31/496; A61K 31/4164; A61K 31/4174; A61K 31/4412; A61K 31/5375; A61K 31/135; A61K 31/27; A61P 17/00; A61K 8/06

(52) U.S. Cl. 424/702; 514/180; 514/254.07; 514/396; 514/399; 514/345; 514/239.5; 514/655; 514/488

(57) ABSTRACT

A foaming gelled composition in the form of a stable oil-in-water microemulsion comprising a fatty phase of microdroplets dispersed in an aqueous phase and a dermatological active ingredient, characterized in that it comprises a surfactant system comprising:—at least one ethoxylated nonionic surfactant;—at least one cosurfactant for stabilizing the ethoxylated nonionic surfactant;—at least one nonethoxylated hydrophilic nonionic surfactant;—at least one foaming agent chosen from the group of anionic surfactants.

The present invention relates to the field of washing foaming liquid forms intended for treating dermatological disorders, in particular disorders of the scalp as well as seborrheic dermatitis.

Seborrheic dermatitis is a dermatological inflammatory chronic affection mainly affecting the head and the face at the sebaceous glands. Seborrheic dermatitis is characterized by marked desquamation at the scalp and at its margin, at the eyebrows as well at the nasalabial fold.

The causes of this affection are not clearly identified. Genetic and hormonal factors would be responsible of this phenomenon. Additionally, a lipophilic yeast of the Pityrosporum genus would be involved in the development of this dermatological disorder.

The treatments of seborrheic dermatitis comprise the use of antifungal compounds such as azolated agents, tars, sulfites or zinc pyrithione for example, so as to control the invasion of Pityrosporum and/or the use of anti-inflammatory agents.

Thus, adequate treatments consist in a daily treatment with an antidandruff shampoo containing selenium sulfite and/or zinc pyrithione. A shampoo based on tars or containing ketoconazole may also be used. Such shampoos should be applied on the scalp and left in place for about 10 minutes without rinsing. The use of a moisturizing or emollient shampoo is recommended in order to limit drying of the scalp. For more severe cases, corticosteroids may be used in a local application.

Patent BE 84515 describes a composition containing hydrocortisone in a mixture of solvents consisting of 15 to 60% of an aliphatic alcohol, 15 to 60% of propylene glycol and 5 to 60% of an additional solubilizer.

Patent EP 325 949 describes a composition comprising at least 2.5% of corticoids, 25 to 80% of a non-ionic surfactant, 0 to 70% of ethanol and 0 to 70% of propylene glycol in association with an antimicrobial agent.

The low fluidity of such compositions makes their application not very easy, all the more so since the solvents which they contain, tend to evaporate rapidly. It is thus necessary to proceed with rubbing in order to allow penetration of the active substances, which causes irritations of the epidermis.

In order to limit this problem of evaporation of the solvent, patent application WO 96/27376 proposes a foam containing a corticoid, a foam-breaking agent, a propellant and a buffer. However, when using it, this type of foam does not allow the scalp to be easily attained.

Such treatments in addition to their low cosmetic attractiveness should be repeated over a rather long time and are accompanied by unpleasant undesirable effects for the patient such as red patches, dryness and irritation of the scalp. The complexity of the treatment makes its observance sometimes limited.

In the case of application of non-water-soluble active ingredients, their solubilization in formulations of the foaming shampoo type is a problem. Further, the time for contact between the foaming formulation and the scalp is limited, which limits all the more penetration of the active substance into the skin.

Thus, emulsion or micro-emulsion formulations of the oil-in-water (O/W) or water-in-oil (W/O) type have been developed for overcoming this kind of drawback. The presence of a lipid phase thus allows better solubilization of the non-water-soluble active ingredients, thus promoting their skin penetration. Further, the emollient properties of the applied oils contribute to the reduction of certain undesirable effects of the treatment (dryness). The time for applying such compositions is also reduced.

Patent U.S. Pat. No. 6,607,733 describes dermatological compositions as O/W micro-emulsions containing an aqueous phase, a discontinuous fatty phase, a stabilizing system associating polyethoxylated and polypropoxylated emulsifiers with bridging substances achieving a bond between the microdroplets of the fatty phase. The presence of these bridging agents is required in order to ensure stability of these emulsions over time. Such a solution is of interest but expensive and technically difficult to apply.

The applicant has searched for compositions which do not have the aforementioned drawbacks, i.e. a composition having good cosmetic properties and allowing easy application and good penetration of the active ingredient.

The applicant has thus developed a foaming composition as a gel of the oil-in-water micro-emulsion type.

The applicant has also noticed that the foaming compositions according to the present invention have the aforementioned properties.

Thus, the compositions according to the present invention allow administration of the active ingredient at the area to be treated, ensure its good penetration into the dermis and also have quite interesting dermatological and cosmetic performances.

With the compositions of the present invention it is possible to treat skin affections such as seborrheic dermitis. The rheological properties of the composition according to the present invention allow them to be easily used, quite comparable with a shampoo. In particular, the presentation as a shampoo with washing and foaming power allows cleaning of the squamas present at the scalp and at the hair. Further, the compositions according to the present invention leave the hair soft and flexible and are not irritating for the scalp.

It was also noticed that the compositions according to the present invention are stable over time and with them it is possible to avoid precipitation of the active ingredient or separation of the phases of the emulsion.

One of the objects of the present invention is therefore a foaming gelled composition of the oil-in-water micro-emulsion type for washing and treating the scalp and hair.

Finally, another object of the present invention is also the use of compositions according to the invention as a drug and for making a drug intended to treat dermatological affections of the skin and of the scalp.

More specifically, the invention therefore relates to a foaming gelled composition as a stable oil-in-water micro-emulsion, comprising a fatty phase of microdroplets dispersed in an aqueous phase and a dermatological active ingredient, characterized in that it comprises a system of surfactants consisting of:
at least one non-ionic ethoxylated surfactant;
at least one co-surfactant stabilizing the non-ionic ethoxylated surfactant;
at least one non-ionic hydrophilic non-ethoxylated surfactant;
at least one foaming agent selected from the group of anionic surfactants.

In the sense of the present invention, by “co-surfactant stabilizing the non-ionic ethoxylated surfactant” is meant a surfactant which places itself between the other surfactant molecules and provides flexibility and fluidity to the interfacial films, thereby allowing stabilization of the interface films between the oil and the water.

In the sense of the present invention, by “foaming agent” is meant a surfactant which may place itself at the water-air interface, this having the effect of stabilizing the formed foam.

Advantageously, the composition according to the invention does not contain any bridging agent. By “bridging agent” is meant a molecule including at least two regions capable of interacting with the oil droplets via a hydrophobic interaction, thereby forming a bridge between two or several oil droplets and a region interacting with the aqueous phase via a hydrophilic interaction.

The active ingredient containing the composition according to the present invention is a dermatological active ingredient, provided with therapeutic virtues towards cutaneous affections of the skin and/or of the scalp.

Within the scope of the present invention, the dermatological active ingredient is for example a non-water-soluble active ingredient preferentially solubilized in the fatty phase of the micro-emulsion.

Corticoids are particularly well adapted as a dermatological active ingredient according to the present invention. The corticoids of the present invention may be selected from the group comprising alclometasone dipropionate, amcinonide, beclomethasone dipropionate, betamethasone benzoate, betamethasone valerate, betamethasone dipropionate, budesonide, clobetasol propionate, preferentially clobetasol 17-propionate, clobetasol butyrate, desonide, desoximetasone, dexamethasone, dediflorasone diacetate, diflucortolone valerate, flurandrenolone, fluprednidene acetate, fluocortolone, fluocortine butyl, fluocinonide, fluocinolone acetonide, fluclorolone acetonide, flumetasone pivalate, feudiline hydrochloride, flumetholone, halcinonide, hydrocortisone, hydrocortisone acetate, hydrocortisone butyrate, hydrocortisone valerate, methylprednisolone acetate, mometasone furoate, methylprednisolone, prednisolone, triamcinolone acetonide or from pharmaceutically acceptable mixtures of the latter.

Advantageously the active ingredient is betamethasone valerate.

Anti-fungal active ingredients are of interest within the scope of the present invention.

Advantageously, the anti-fungal agents are selected from the group comprising imidazoles, non-imidazolated pyridones, morpholines, allylamines.

The imidazole by advantageously be selected from the group comprising ketoconazole, bifonazole, clotrimazole, econazole, fenticonazole, isoconazole, miconazole, omoconazole, oxiconazole, sulconazole, tioconazole.

The non-imidazolated pyridone is advantageously cyclopiroxolamine.

Advantageously the morpholine is amorolfine.

The allylamine may be selected from the group comprising terbinafine and tolnaftate.

Anti-seborrheic active ingredients, such as for example selenium sulfide and benzoyl peroxide, may be used according to the present invention.

The amount of dermatological active ingredient is comprised by weight between 0.001% and 5%, advantageously between 0.01% and 1%, still more advantageously between 0.05% and 1%, even more advantageously about equal to 0.1% of the total weight of the composition.

The aqueous phase may contain in addition to water, cosmetically acceptable solvents such as for example glycerol, propylene glycol, sorbitol, ethanol, polyethylene glycols, isopropanol, one or more water-soluble compounds such as acids or bases, coloring agents, moisturizing, soothing, healing, exfoliating, antioxidant active agents, or any other additive or adjuvant not detrimental to the stability of the micro-emulsions.

The aqueous phase may also contain one or more water-soluble compounds such as acids or bases, coloring agents, moisturizing, soothing, healing, exfoliating, antioxidant active agents or any other additive or adjuvant not detrimental to the stability of the micro-emulsions.

The amount of the aqueous phase of the composition according to the present invention is advantageously comprised by weight between 30% and 60%, more advantageously between 35% and 55% of the total weight.

The fatty phase of the composition has all its importance within the scope of the present invention. Indeed, the micro-droplets of this fatty phase (discontinuous phase of the micro-emulsion) are used for solubilizing the non-water-soluble active ingredient and for carrying the latter at the scalp, thereby ensuring its vectorization in the dermis. Thus, the bioavailability of the composition according to the present invention is highly improved as compared with a composition for which the active ingredient would be found as dispersed particles with a very limited transcutaneous passage.

Further, as indicated in the introduction of the present application, the skin dryness problems encountered with the compositions of the prior art may be limited or inhibited. Indeed, the fatty phase of the composition according to the invention has emollient properties with which the skin and the scalp may be preserved.

The amount of fatty phase in the composition according to the present invention is advantageously comprised by weight between 0.5% and 20%, more advantageously between 2 and 18%, still more advantageously between 2.5% and 15%, and still more advantageously between 5% and 10% of the total weight of the composition.

The components of the fatty phase may be selected from the group comprising vegetable or mineral oils, triglycerides, monoglycerides, diglycerides, fatty acid derivatives, fatty acid esters, cyclic or aliphatic, linear or branched (either saturated or not) hydrocarbon natural oils either hydrogenated or not, either synthetic or not, silicones either volatile or not, either organomodified or not, either soluble or not, perfluorinated or fluorinated oils, polybutene and polyisobutene, fatty acids existing as liquids, either used alone or in a mixture. For example, this may be a paraffinic oil, octododecyl myristate, isopropyl myristate, tricaprylic glycerol, capric-caprylic glyceride, either used alone or as a mixture.

By organomodified silicone is meant a silicone including one or more functional groups attached onto the siloxane chain, optionally via a hydrocarbon radical. As an example, mention is made of such groups as polyethyleneoxy, polypropyleneoxy, optionally including alkyl groups such as alkyl methicone copolyol sold by Dow Corning under the designation Q2 5200.

The non-ionic ethoxylated surfactant may be selected from the group comprising ethoxylated fatty alcohols, ethoxylated fatty acids, ethoxylated fatty acids etherified by alkyl or alkenyl groups, ethoxylated fatty acids esterified by alkyl or alkenyl groups, ethoxylated esters of saturated or unsaturated fatty acids, of glycerol, of propylene glycol, of glycol, of polyethylene glycol, of sorbitan, the mixtures of glycerides and caprylic esters of polyethylene glycol, copolymers of ethylene oxide and propylene oxide (of the Poloxamer® of Pluronic® type).

The amount of non-ionic ethoxylated surfactant present in the composition according to the present invention is advantageously less than 20% by weight of the total weight of the composition, more advantageously comprised between 10 and 20% of the total weight.

In order to stabilize the micro-emulsion according to the present invention, the present invention comprises at least one co-surfactant stabilizing the non-ionic ethoxylated surfactant. The amount of stabilizer will be defined relatively to the non-ionic ethoxylated surfactant. The co-surfactant:non-ionic ethoxylated surfactant ratio may advantageously be comprised by weight between 1:2 and 1:6, more advantageously between 1:2 and 1:4, this depending on the nature of both the surfactant and the non-ionic ethoxylated surfactant.

The total amount of non-ionic ethoxylated surfactant and of stabilizing co-surfactant is advantageously comprised by weight between 15% and 40%, more advantageously between 20% and 35% of the total weight of the composition.

The co-surfactant stabilizing the non-ionic ethoxylated surfactant in a composition according to the present invention may be selected from the group comprising non-ethoxylated esters of saturated or unsaturated fatty acids and of glycerol, of polyglycerol, of propylene glycol, of glycol, of polyethylene glycol or of sorbitan, such as triglycerol diisostearate, triglycerol oleate, propylene glycol monolaurate, propylene glycol monocaprylate, sorbitan monostearate.

The amount by weight of non-ethoxylated hydrophilic non-ionic surfactant is advantageously comprised by weight between 1 and 30%, more advantageously between 10% and 30%, still more advantageously between 13% and 25% of the total weight.

The suitable non-ethoxylated hydrophilic non-ionic surfactants for the application of the present composition may be selected from surfactants derived from sugars, fatty acid and sucrose, saccharose or glucose esters.

Advantageously, the non-ethoxylated hydrophilic non-ionic surfactants may be selected from the group comprising glucoside derivatives, such as alkylglucosides and alkylpolyglucosides, having C₈-C₂₂ carbon chains, with numbers of glucose units from 1 to 7, for example octyl glucoside, decyl glucoside, lauryl glucoside, myristyl glucoside, palmityl glucoside, stearyl glucoside, oleyl glucosides, behenyl glucosides; in particular glucoside derivatives having C₁₀-C₁₄ carbon chains with a number of glucose units from 1 to 3 such as decyl glucoside (Oramix NS10, Seppic).

The pH of the compositions according to the invention is advantageously comprised between 3 and 8, more advantageously between 4 and 6. Selection of the pH will depend on the retained active ingredient and on its chemical structure.

The pH is advantageously adjusted by adding lactic acid.

The composition according to the present invention also comprises an anionic foaming agent, in order to reinforce the foaming and cleaning functionality useful for the desired applications of the present invention. The foaming agent according to the present invention may be selected from the group of anionic surfactants comprising the sodium, ammonium, amine, amino-alcohol or magnesium salts of the following compounds: alkylsulfates, alkylethersulfates, alkylamidoether sulfates, alkylarylpolyether sulfates, alkyl monoglyceride sulfates, alkyl monoglyceride ether sulfates, alkyl monoglyceride sulfonates, alkyl sulfonates, alkyl phosphates, alkyl isethionates, alkyl carboxylates, alkyl ether carboxylates, alkyletheramido carboxylates, alkylamide sulfonates, alkylaryl sulfonates, α-olefin sulfonates, paraffin sulfonates, alkylsulfosuccinates, alkylethersulfo-succinates, alkylamidosulfosuccinates, alkylsulfo-succinamates, alkylsulfo-acetates, alkyletherphosphates, acyl sarcosinates, N-acyl aminoacids, acylisethioates and N-acyltaurates.

Advantageously, the amount by weight of anionic foaming agent is comprised between 15% and 25% of the total weight.

The composition according to the present invention may further comprise adjuvants traditionally used in dermatological or cosmetic compositions such as shampoos. The present composition may thus comprise foam-stabilizing or foam-reinforcing agents such as fatty acid amides, alkyl amides, mono- and di-ethanol amides, isopropanol amides of fatty acids.

Advantageously, the foam-stabilizing or foam-reinforcing agent will be coprah fatty acid diethanolamide, for example Comperlan KD (Cognis).

The amount of foam-stabilizing or foam-reinforcing agent in the present composition may advantageously be comprised by weight between 0% and 10%, advantageously between 2% and 8%, for example 6% by weight based on the total weight of the composition.

The composition according to the present invention thus exists as an oil-in-water micro-emulsion, in which the non-water-soluble dermatological active ingredient is found at the fatty phase. This fatty phase is stabilized by means of the surfactant and co-surfactant system applied in the present composition.

In order to gel the composition according to the present invention for better practical use, this composition may contain thickeners, notably selected from non-bridging agents such as electrolytes (advantageously sodium chloride or sodium citrate), carbomers or xanthan gums.

The amount of thickeners in the present invention may be comprised by weight between 0 and 4%, advantageously between 1 and 3% of the total weight of the composition.

By adding NaCl to the present composition it is possible to advantageously increase the viscosity of said composition. The present composition may thus comprise up to 2%, advantageously 1% of NaCl by weight, based on the total weight of the composition.

Thus, within the scope of the present invention, the compositions are in the form of liquids, particularly in the form of viscous liquids.

In order to protect the composition according to the present invention from risks of microbial contamination, one or more preservatives may be added. The preservatives suitable for application of the present composition may be selected from conventional preservatives of drugs for topical use such as acids and their salts (boric acid, benzoic acid, lactic acid, sorbic acid), alcohols (benzyl alcohol, phenoxyethanol, chlorocresol), (methyl, ethyl, propyl, butyl, paraben) esters of parahydroxybenzoic acid and imidazolidinurea derivatives. Advantageously, preservatives will be selected from the group comprising sorbic acid and its salts, in particular potassium sorbate used at a concentration from 0.01 to 0.2% by weight.

The compositions according to the invention are more particularly used as a shampoo for treating the scalp. When the selected active ingredient is betamethasone valerate, the composition according to the invention is more particularly intended for treating seborrheic dermatitis of the skin in general and of the scalp in particular.

When using it, the composition is applied on wet hair. It is proceeded with a slight massage during which a foam is formed, followed by careful rinsing after having observed a pause. It may be proceeded with a second application followed by careful rinsing again with water.

The emollient effect of the composition according to the present invention makes its use particularly suitable for treating dermatological disorders such as seborrheic dermatitis. Indeed, as this affection is characterized by red patches and irritations, with the use of the composition according to the present invention it is possible to limit the traditionally observed skin dryness phenomena.

The compositions according to the present invention may be made according to conventional techniques of the pharmaceutical and cosmetical industry.

Thus, in a first phase, it is proceeded with intimate mixing of the fatty phase with the different surfactants and the co-surfactant. Said mixture may be subject to homogenization by mechanical stirring before emulsification.

The aqueous phase containing the optional water-soluble adjuvants such as for example NaCl, will be prepared separately and subsequently incorporated into the fatty phase.

The step for incorporating the aqueous phase into the fatty phase is carried out with stirring, at a rate and for sufficient time in order to reach transparence of the composition.

The pH of the thereby obtained micro-emulsion may be adjusted via the addition of an acid or basic solution. After adjusting the pH, the incorporation of the active ingredient may be carried out by stirring until complete dissolution at room temperature. As an example, in the case of the use of betamethasone valerate, the use of lactic acid for adjusting the pH will be preferred.

Therefore, the object of the invention is also a method for preparing a composition according to the invention, comprising the following successive steps:
mixing the fatty phase with the surfactant system and homogenizing the mixture by mechanical stirring;
mixing the different adjuvants in the aqueous phase, such as for example the thickener;
incorporating the aqueous phase into the fatty phase with stirring, at a rate and for sufficient time in order to attain transparence of the composition;
optionally adjusting the pH of the emulsion by adding an acid or basic solution;
incorporating the active ingredient by stirring until complete dissolution of the latter at room temperature.

The object of the invention is also a composition as described earlier, as a drug.

The object of the invention is also the use of a composition as described earlier for making a drug intended for treating dermatological disorders, the scalp and seborrheic dermatitis.

The object of the invention is also the use of a composition as described earlier as a cosmetic product.

The invention will now be illustrated in a non-limiting way by the following examples.

Ingredients:
Texapon N 70® and Texapon NSO® sodium laureth sulfate were obtained from Cognis.
Ondina 68® and Primol 352® mineral oils were obtained from Shell and Exxon Mobil respectively.
PEG-40 Labrasol® hydrogenated castor oil was obtained from Gatterfosse.
MEA Comperlan KD® cocamide was obtained from Cognis.
Oramix NS 10® decylglucoside was obtained from Seppic.
Oleic plurol and diisostearic plurol were obtained from Gattefosse.

### EXAMPLE 1

Two micro-emulsions without any active ingredient (bases) were prepared and analyzed. Their compositions are indicated in Table 1 and their analyses are gathered in Table 2. The percentages indicate a concentration by weight of the corresponding product in solution in water.

Both emulsions are stable, transparent and have good sensorial analysis and proper viscosity.

TABLE 1
Formulation of micro-emulsions without any
active ingredient.

| Bases | Ingredients | Proportions |
|---|---|---|
| 1 | Ondina 68 | 5% |
| | Diisostearic plurol/labrasol | 18% |
| | ⅓ | |
| | 10% decylglucoside | 20% |
| | 6% Texapon NSO | 20% |
| | 6% Comperlan KD | 6% |
| | NaCl | 1% |
| 2 | Ondina 68 | 5% |
| | Oleic plurol/labrasol ½ | 21.5% |
| | 12.5% decylglucoside | 25% |
| | 5% Texapon NSO | 16.5% |
| | 2% Comperlan KD | 2% |
| | NaCl | 1% |

The foaming power is obtained by measuring the foam height after churning for 90 seconds.

The resistance to dirt is a measurement of the foam height after churning for 90 seconds and incorporating one or several milliliters of artificial dirt. The emulsion to be analyzed (40 mL) was placed in a standardized test tube. The dirt consists of 5% lanolin, 5% paraffin oil, and dioxane qsp 100%. 1 mL of dirt was added to the emulsion. The solution was stirred for 90 seconds and the foam height H1 was then measured. After 20 seconds at rest, 1 mL of dirt was again added, the solution was stirred for 30 seconds and the foam height H2 was measured.

The wetting power is measured according to a method adapted from the standards AFNOR NF 73-406 and NF-73-420, consisting of measuring the time required for the emulsion to completely wet a standardized cotton disc with a diameter of 3 cm. The measurements were taken for different surfactant concentrations and the wetting power corresponds to the surfactant concentration for which the wetting time is 100 seconds.

TABLE 2
Analysis of micro-emulsions without any active
ingredient

| | Base 1 | Base 2 |
|---|---|---|
| Aspect | Pale yellow | Colorless |
| pH | 5.44 | 5.67 |
| Viscosity (Pa · s), | 0.212 | 0.220 |
| D = 100 s⁻¹ | | |
| Wetting power | 5.0 | 4.5 |
| (g/L) | | |
| Foaming power (cm) | H0 = 14.9 | H0 = 15 |
| Dirt resistance | H2 = 0.6 | H1 = 1.1 |
| (cm) | | H2 = 0.3 |
| Amount of foam | Large | Very large |
| | (rapid foam | |
| | onset) | |
| Foam stability | Good | Very good |
| Rinsing | Good | Good |
| Disentangling | Very good | Good |

### EXAMPLE 2

Two micro-emulsions comprising an active ingredient were prepared. The viscosity was improved relatively to that of the bases but increasing the NaCl content. Texapon NSO was replaced with Texapon N70, the pH was adjusted with lactic acid. The formulations are indicated in Table 3.

TABLE 3
Emulsions comprising an active ingredient.

| | Formula 1 | Formula 2 | Formula 3 |
|---|---|---|---|
| | (%) | (%) | (%) |
| Oramix NS10 | 22.73 | 18.18 | 1 |
| Oramix L30 | | | 10 |
| Texapon N70 | 7.142 | 8.57 | 30 |
| Comperlan KD | 2 | 6 | |
| Labrasol | 15.66 | 13.5 | 15 |
| Oleic plurol | 7.83 | | |
| Diisostearic | | 4.5 | 5 |
| plurol | | | |
| Primol 352 | 5 | 5 | |
| HP Ondina | | | 5 |
| NaCl | 1 | 1 | |
| Water | 38.53 | 43.01 | 34 |
| Lactic acid | 0.0125 | 0.14 | Qsp pH |
| | | | 5.5 |
| Bethamethasone | 0.1 | 0.1 | |
| valerate | | | |
| Cyclopyroxolamine | | | 1 |
| pH without any | 5.52 | 5.65 | |
| active ingredient | | | |
| pH with active | 5.37 | 5.47 | |
| ingredient | | | |
| Aspect | Transparent | Transparent | |
| | yellow micro- | pale yellow | |
| | emulsion | micro- | |
| | | emulsion | |

1. A foaming gelled composition as a stable oil-in-water micro-emulsion, comprising a fatty phase of micro-droplets dispersed in an aqueous phase and a dermatological active ingredient, characterized in that it comprises a surfactant system consisting of:
at least one non-ionic ethoxylated surfactant;
at least one co-surfactant stabilizing the non-ionic ethoxylated surfactant;
at least one non-ionic hydrophilic non-ethoxylated surfactant;
at least one foaming agent selected from the group of anionic surfactants.

2. The composition according to claim 1, characterized in that the active ingredient is provided with therapeutic virtues towards cutaneous affections of the skin and/or of the scalp.

3. The composition according to claim 1, characterized in that the active ingredient is dissolved in the fatty phase.

4. The composition according to claim 1, characterized in that the active ingredient is a corticoid which may be selected from the group comprising alclometasone dipropionate, amcinonide, beclomethasone dipropionate, betamethasone benzoate, betamethasone valerate, betamethasone dipropionate, budesonide, clobetasol propionate, preferentially clobetasol 17-propionate, clobetasol butyrate, desonide, desoximetasone, dexamethasone, dediflorasone diacetate, diflucortolone valerate, flurandrenolone, fluprednidene acetate, fluocortolone, fluocortine butyl, fluocinonide, fluocinolone acetonide, fluclorolone acetonide, flumetasone pivalate, feudiline hydrochloride, flumetholone, halcinonide, hydrocortisone, hydrocortisone acetate, hydrocortisone butyrate, hydrocortisone valerate, methylprednisolone acetate, mometasone furoate, methylprednisolone, prednisolone, triamcinolone acetonide or from pharmaceutically acceptable mixtures of the latter and is preferably betamethasone valerate.

5. The composition according to claim 1, characterized in that the active ingredient is an antifungal agent which may be selected from the group comprising imidazoles, non-imidazolated pyridones, morpholines, allylamines.

6. The composition according to claim 5, characterized in that the imidazole may be selected from the group comprising ketoconazole, bifonazole, clotrimazole, econazole, fenticonazole, isoconazole, miconazole, omoconazole, oxiconazole, sulconazole, tioconazole.

7. The composition according to claim 5, characterized in that the non-imidazolated pyridine is cyclopiroxolamine.

8. The composition according to claim 5, characterized in that the morpholine is amorolfine.

9. The composition according to claim 5, characterized in that the allylamine may be selected from the group comprising terbinafine and tolnaftate.

10. The composition according to claim 1, characterized in that the active ingredient may be an anti-seborrheic agent preferably selenium sulfide or benzoyl peroxide.

11. The composition according to claim 1, characterized in that the amount of dermatological active ingredient is comprised by weight between 0.001% and 5%, preferentially between 0.01% and 1%, still more preferentially between 0.05% and 1%, even more preferentially about equal to 0.1% of the total weight.

12. The composition according to claim 1, characterized in that the aqueous phase further comprises one or more cosmetically acceptable solvents which may be selected from the group comprising glycerol, propylene glycol, sorbitol, ethanol.

13. The composition according to claim 1, characterized in that the aqueous phase further comprises one or more water-soluble compounds which may be selected from the group comprising acids, bases, coloring agents, moisturizing, soothing, healing, exfoliating, antioxidant active agents.

14. The composition according to claim 1, characterized in that the amount of aqueous phase is comprised by weight between 30% and 60%, preferentially between 35% and 55% of the total weight.

15. The composition according to claim 1, characterized in that the amount of fatty phase is comprised by weight between 0.5% and 20%, more preferentially between 2 and 18%, still more preferentially between 2.5% and 15%, and even more preferentially between 5% and 10% of the total weight.

16. The composition according to claim 1, characterized in that the fatty phase consists of constituents which may be selected from the group comprising vegetable or mineral oils, triglycerides, monoglycerides, diglycerides, fatty acid derivatives, fatty acid esters, natural hydrocarbon, cyclic or aliphatic, linear or branched oils either hydrogenated or not, either synthetic or not, either saturated or not, silicones either volatile or not, either organomodified or not, either soluble or not, perfluorinated or fluorinated oils, polybutenes and polyisobutenes, liquid fatty acids either used alone or in a mixture, paraffinic oils, octododecyl myristate, isopropyl myristate, tricaprylic glycerol, capric-caprylic glyceride.

17. The composition according to claim 1, characterized in that the non-ionic ethoxylated surfactant may be selected from the group comprising ethoxylated fatty alcohols, ethoxylated fatty acids, ethoxylated fatty acids etherified by alkyl or alkenyl groups, ethoxylated fatty acids esterified by alkyl or alkenyl groups, ethoxylated esters of saturated or unsaturated fatty acids, of glycerol, of propylene glycol, of glycol, of polyethylene glycol, of sorbitan, the mixtures of glycerides and caprylic esters of polyethylene glycol, copolymers of ethylene oxide and propylene.

18. The composition according to claim 1, characterized in that the amount by weight of non-ionic ethoxylated surfactant present in the composition is less than 20% of the total weight of the composition, preferentially comprised between 10 and 20% of the total weight.

19. The composition according to claim 1, characterized in that the ratio of the amount by weight of cosurfactant stabilizing the non-ionic ethoxylated surfactant over the amount of non-ionic ethoxylated surfactant is comprised between 1:2 and 1:6, preferentially between 1:2 and 1:4.

20. The composition according to claim 1, characterized in that the total amount by weight of non-ionic ethoxylated surfactant and of the co-surfactant stabilizing the non-ionic ethoxylated surfactant is comprised by weight between 15% and 40%, preferentially between 20% and 35% of the total weight.

21. The composition according to claim 1, characterized in that the co-surfactant stabilizing the non-ionic ethoxylated surfactant may be selected from the group comprising non-ethoxylated esters of saturated or unsaturated fatty acids and of glycerol, of polyglycerol, of propylene glycol, of glycol, of polyethylene glycol or of sorbitan, triglycerol diisostearate, triglycerol oleate, propylene glycol monolaurate, propylene glycol monocaprylate, sorbitan monostearate.

22. The composition according to claim 1, characterized in that the amount by weight of non-ethoxylated hydrophilic non-ionic surfactant is comprised between 1 and 30%, preferably between 10% and 30%, more preferentially between 13% and 25% of the total weight.

23. The composition according to claim 1, characterized in that the non-ethoxylated hydrophilic non-ionic surfactant may be selected from the group comprising sugar derivatives, fatty acid and sucrose, saccharose or glucose esters, glucoside derivatives, alkylglucosides, C₈-C₂₂ alkylpolyglucosides comprising 1 to 7 glucose units, octyl glucoside, decyl glucoside, lauryl glucoside, myristyl glucoside, palmityl glucoside, stearyl glucoside, oleyl glucosides, behenyl glucoside, C₁₀-C₁₄ glucoside derivatives comprising 1 to 3 glucose units, decyl glucoside.

24. The composition according to claim 1, characterized in that its pH is comprised between 3 and 8, preferentially between 4 and 6.

25. The composition according to claim 1, characterized in that its pH is adjusted by adding lactic acid.

26. The composition according to claim 1, characterized in that the anionic foaming agent may be selected from the group comprising sodium, ammonium, amine, amino alcohol, or magnesium salts of alkyl sulfates, alkylether sulfates, alkylamidoether sulfates, alkylarylpoly-ether sulfates, alkylmonoglyceride sulfates, alkylmonoglycerideehter sulfates, alkylmonoglyceride sulfonates, alkyl sulfonates, alkyl phosphates, alkyl isethionates, alkyl carboxylates, alkylether carboxylates, alkylether amidocarboxylates, alkylamide sulfonates, alkylaryl sulfonates, α-olefin sulfonates, paraffin sulfonates, alkylsulfo succinates, alkylethersulfo succinates, alkylamidesulfo succinates, alkylsulfo succinamates, alkylsulfo acetates, alkylehter phosphates, acylsarconsinates, N-acyl amino acids, acylisothionates, and N-acyltaurates.

27. The composition according to claim 1, characterized in that the amount of anionic foaming agent by weight is comprised between 15% and 25% of the total weight.

28. The composition according to claim 1, characterized in that it further comprises at least one foam stabilizing or reinforcing agent.

29. The composition according to claim 28, characterized in that the stabilizing or reinforcing agent may be selected from the group comprising fatty acid amides, alkyl amides, mono- and di-ethanol amides, fatty acid isopropanol amides and are preferably coprah fatty acid diethanol amide.

30. The composition according to claim 28, characterized in that the amount by weight of foam stabilizing or reinforcing agent is comprised between 0% and 10%, preferentially between 2% and 8% of the total weight.

31. The composition according to claim 1, characterized in that it further comprises a thickener selected from non-bridging agents.

32. The composition according to claim 31, characterized in that the thickener may be selected from the group comprising sodium salts, carbomers, or xanthan gums, and is preferably sodium chloride or sodium citrate.

33. The composition according to claim 31, characterized in that the amount by weight of thickener is comprised between 0% and 4%, preferentially between 1% and 3% of the total weight.

34. The composition according to claim 1, characterized in that it further comprises a preservative.

35. The composition according to claim 34, characterized in that the preservative may be selected from the group comprising boric acid, benzoic acid, lactic acid, sorbic acid, benzyl alcohol, phenoxy ethanol, chlorocresol, esters of parahydroxy benzoic acid, derivatives of imidazolidinurea and is preferentially sorbic acid and its salts, and more preferentially potassium sorbate.

36. The composition according to claim 34, characterized in that the preservative is potassium sorbate at a weight concentration comprised between 0.01% and 0.2% of the total weight.

37. A method for preparing a composition according to claim 1, characterized in that it comprises the following successive steps:
mixing the fatty phase with the surfactant system and homogenizing the mixture with mechanical stirring;
mixing the different adjuvants in the aqueous phase, such as for example the thickener;
incorporating the aqueous phase into the fatty phase with stirring at a rate and for a sufficient time in order to attain transparency of the composition;
optionally adjusting the pH of the emulsion by adding an acid or basic solution;
incorporating the active ingredient with stirring until complete dissolution of the latter at room temperature.

38. The composition according to claim 1 as a drug.

39. The use of a composition according to claim 1 for making a drug intended for treating dermatological disorders, the scalp and seborrheic dermatitis.

40. The use of a composition according to claim 1 as a cosmetic product.
